(19) United States
(12) Patent Application Publication
(10) Pub. No.: US 2024/0344107 A1
(43) Pub. Date: Oct. 17, 2024

(54) Bacteria Detection Device and Bacteria Detection Method

(71) Applicants: SHIMADZU CORPORATION; Hyogo Medical University, Nishinomiya-shi (JP); Kyoto University, Kyoto-shi (JP)

(72) Inventors: Hiroshi IWATA, Kyoto-shi (JP); Hiroomi GOTO, Kyoto-shi (JP); Shojiro KIKUCHI, Nishinomiya-shi (JP); Yuichi OGAWA, Kyoto-shi (JP)

(21) Appl. No.: 18/606,784

(22) Filed: Mar. 15, 2024

(30) Foreign Application Priority Data
Mar. 17, 2023 (JP) 2023-043013

(51) Int. Cl. C12Q 1/18; C12M 1/00; C12M 1/12; C12M 1/34

(52) U.S. Cl. CPC C12Q 1/18; C12M 25/04; C12M 29/04; C12M 29/06; C12M 41/36

(57) ABSTRACT

The bacteria detection device includes a reservoir which stores a bacterial solution containing bacteria, a sensor which is provided on a bottom of the reservoir and configured to detect the bacteria in the bacterial solution stored in the reservoir, and a filter which is placed on a liquid surface of the bacterial solution stored in the reservoir to permeate the bacterial solution. The filter increases the concentration of the bacteria in the bacterial solution present in a detection region of the sensor by permeating the bacterial solution.

### CROSS REFERENCE TO RELATED APPLICATIONS

This non-provisional application is based on Japanese Patent Application No. 2023-043013 filed on Mar. 17, 2023 with the Japan Patent Office, the entire contents of which are hereby incorporated by reference.

### BACKGROUND OF THE INVENTION

### Field of the Invention

The present disclosure relates to a bacteria detection device and a bacteria detection method.

### Description of the Background Art

As a conventional bacteria detection device, there is one that detects the presence of viable bacteria such as Mycobacterium tuberculosis and Escherichia coli. Such bacteria detection device includes one that detects bacteria using a CMOS oscillator sensor.

If bacteria are present in a bacterial solution (a culture solution containing bacteria and liquid), a dielectric constant of the bacterial solution will change. For example, a conventional bacteria detection device equipped with a CMOS oscillator sensor detects a dielectric constant of the bacterial solution, and determine the presence or absence of bacteria in the bacterial solution according to the detected dielectric constant of the bacterial solution (Y. Ogawa, et al., Near-field sensor array with 65-GHz CMOS oscillators for rapid detection of viable Escherichia coli, Biosensors and Bioelectronics, 176 (2021) 112935).

### SUMMARY OF THE INVENTION

When the sensor detects bacteria in the bacterial solution according to the detected dielectric constant of the bacterial solution, the following problem may occur. For example, if the depth of the bacterial solution stored in a reservoir such as a well exceeds a detection region of the sensor in the depth direction of the reservoir, the sensor cannot detect the bacteria in the bacterial solution beyond the detection region. Thus, it is difficult for the sensor to accurately detect the bacteria in the bacterial solution.

It is an object of the present disclosure is to enable a sensor to accurately detect bacteria in a bacterial solution.

A bacteria detection device according to an aspect of the present disclosure includes a reservoir which stores a bacterial solution containing bacteria and liquid, a sensor which is provided on a bottom of the reservoir and configured to detect the bacteria contained in the bacterial solution stored in the reservoir, and a filter which is placed on a liquid surface of the bacterial solution stored in the reservoir and has an opening for selectively permeating the liquid in the bacterial solution. The filter is configured to selectively permeate the liquid in the bacterial solution, thereby increasing the concentration of the bacteria contained in the bacterial solution in a detection region of the sensor.

A bacteria detection method according to another aspect of the present disclosure includes bringing a bacterial solution containing bacteria and liquid into contact with a sensor configured to detect the bacteria, and selectively removing the liquid in the bacterial solution out of a detection region of the sensor by using a filter having an opening to selectively permeate the liquid in the bacterial solution so as to increase the concentration of the bacteria contained in the bacterial solution in the detection region of the sensor.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a diagram illustrating a configuration of a bacteria detection device 1 according to a first embodiment;

FIG. 2 is a cross-sectional view illustrating operation states of a storage member 3 relative to a reservoir 2 during a bacteria detection by the bacteria detection device 1 according to the first embodiment;

FIG. 3 is a plan view illustrating a multi-well plate 20 to be detected by the bacteria detection device 1 according to the first embodiment;

FIG. 4 is a diagram illustrating an example configuration of the bacteria detection device 1 which is used to perform a bacteria detection on a plurality of reservoirs 2 according to the first embodiment;

FIG. 5 is a flowchart illustrating steps of a bacteria detection method;

FIG. 6 is a cross-sectional view illustrating operation states of a storage member 6 relative to the reservoir 2 during a bacteria detection by a bacteria detection device 1A according to a second embodiment;

FIG. 7 is a illustrating a multi-well plate 20 used in the drug sensitivity detection;

FIG. 8 is a graph illustrating a detection result when Mycobacterium tuberculosis is present in the bacterium solution; and

FIG. 9 is a graph illustrating a detection result when Mycobacterium tuberculosis is not present in the bacterial solution.

### DESCRIPTION OF THE PREFERRED EMBODIMENTS

Hereinafter, embodiments of the present invention will be described in detail with reference to the accompanying drawings. In the drawings, the same or corresponding parts are denoted by the same reference numerals, and the description thereof will not be repeated. In the following description, a plurality of embodiments will be described, and appropriate combinations of components described in the respective embodiments are also originally intended.

In the following embodiments, a bacterial solution 23 contains bacteria 7 and liquid, and a bacteria detection device will be described as a basic configuration in which a filter 32 placed on the bacterial solution 23 is used to selectively permeate the liquid in the bacterial solution 23 so as to increase the concentration of the bacteria 7 of the bacterial solution 23 in a detection region of a sensor 22.

### First Embodiment

In the first embodiment, in addition to the basic configuration described above, a bacteria detection device 1 stores the liquid selectively permeated through the filter 32 from the bacterial solution 23 in a cylindrical storage member 3 provided above the filter 32, whereby the liquid selectively permeated through the filter 32 from the bacterial solution 23 is stored in the storage member 3.

### [Overall Configuration of Bacteria Detection Device 1]

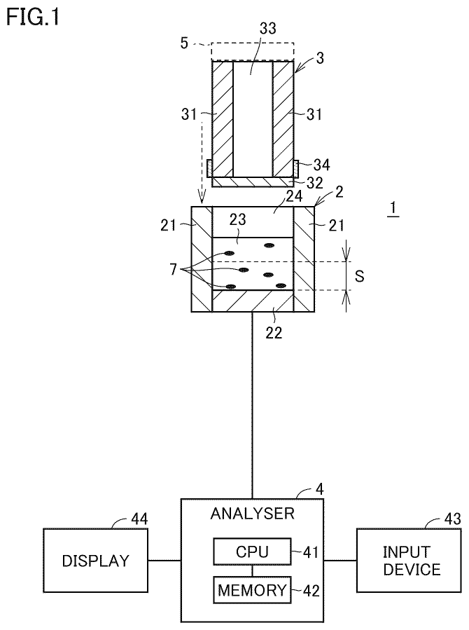

FIG. 1 is a diagram illustrating a configuration of a bacteria detection device 1 according to a first embodiment. The configuration of the bacteria detection device 1 will be described with reference to FIG. 1. The bacteria detection device 1 includes a reservoir 2, a sensor 22, a filter 32, a storage member 3, an analyzer 4, an input device 43, and a display 44.

In FIG. 1, the reservoir 2, the sensor 22, the filter 32, and the storage member 3 are illustrated in cross-sectional view. The reservoir 2 is a cylindrical well. The reservoir 2 is formed of a transparent resin, a top end thereof is opened, and a lower bottom thereof is provided with a disk-shaped sensor 22. In order to store a bacterium liquid 23 which is a culture solution containing bacteria 7 and liquid, the bottom of the reservoir 2 is sealed.

When a detection is performed on cultured viable bacteria such as Mycobacterium tuberculosis or Escherichia coli (hereinafter, referred to as a bacteria detection) by using the bacteria detection device 1, the bacterial solution 23 containing the bacteria 7 and the liquid is injected into the reservoir 2 from the top end thereof. The injected bacterial solution 23 is stored in the reservoir 2 from the bottom where the sensor 22 is provided toward the upper side along the wall 21.

The sensor 22 is constituted by a CMOS oscillator sensor, and detects the bacteria 7 in the bacterial solution 23 according to a change in the dielectric constant of the bacterial solution 23. The detection region (detection range) S of the sensor 22 inside the reservoir 2 is limited to a range of, for example, 10 μm to 20 μm upward from the upper surface of the sensor 22. The detection region of the sensor 22 is also referred to as a sensitivity region.

In FIG. 1, the size ratio of each part such as the width of the detection region S, the thickness of the sensor 22, the size of the reservoir 2, the thickness of the filter 32, the size of the bacteria 7, and the size of the storage member 3 is different from the actual size ratio in order to clearly show the technical characteristics of the bacteria detection device 1.

The storage member 3 is a cylindrical body having an outer diameter slightly smaller than an inner diameter of the reservoir 2, a top end thereof is open, and a lower bottom thereof is attached with a disc-shaped filter 32 having the same diameter as the outer diameter of the storage member 3. The filter 32 is a hydrophilic membrane filter that has a large number of openings such as pores with a minute diameter and is configured to allow the liquid in the bacterial solution 23 to selectively permeate therethrough but not to allow the bacteria 7 to permeate therethrough. The filter 32 is attached to a frame that will not deform when the liquid is selectively permeated through the filter 32 from the bacterial solution 23.

It should be noted that the filter 32 may be configured not to allow the bacteria 7 to permeate therethrough at all, or may be configured to be basically difficult for the bacteria 7 to permeate therethrough but allow the minute bacteria 7 to permeate therethrough. An example size of the bacteria 7 is 0.5 to 10 μm. Regarding the bacteria 7 having the mentioned size, an example pore size of the filter 32 is 0.1 to 0.45 μm. The size of the bacteria 7 and the pore size of the filter 32 are not limited to the example size mentioned above.

When the bacteria detection device 1 is used to perform a bacteria detection, as indicated by a broken arrow in the figure, the storage member 3 is coaxially inserted into an internal space 24 of the reservoir 2. A pusher member 5 is attached to a top end of the storage member 3 so as to gradually push the storage member 3 at a constant speed in the internal space 24 of the reservoir 2. The pusher member 5 is attachable to or detachable from the storage member 3.

In the storage member 3, a sealing member 34 made of an O-ring or the like is provided on the outer periphery of a lower bottom portion of the wall 31. Thus, even when the storage member 3 is inserted into the internal space 24 of the reservoir 2 and pushed downward with the filter 32 being placed on the bacterial solution 23 in the reservoir 2, the sealing member 34 prevents the bacterial solution 23 from leaking upward from between the outer periphery of the wall 31 of the storage member 3 and the inner periphery of the wall 21 of the reservoir 2.

When the storage member 3 is inserted into the internal space 24 of the reservoir 2 along the wall 21 of the reservoir 2, the filter 32 is placed on the liquid surface of the bacterial solution 23 stored in the internal space 24 of the reservoir 2. When the filter 32 comes into contact with the bacterial solution 23, the bacterial solution 23 in the reservoir 2 permeates through the filter 32 of the storage member 3.

When the filter 32 is placed on the liquid surface of the bacterial solution 23, the storage member 3 is gradually pushed downward by the weight of the pusher member 5. When the storage member 3 is gradually pushed downward in this manner, the liquid in the bacterial solution 23 stored in the internal space 24 of the reservoir 2 selectively permeates through the filter 32 and flows into an internal space 33 of the storage member 3 in response to a downward force applied to the filter 32. In this state, the internal space 33 surrounded by an inner surface of the wall 31 and an upper surface of the filter 32 forms a space to store the liquid selectively permeated through the filter 32 from the bacterial solution 23.

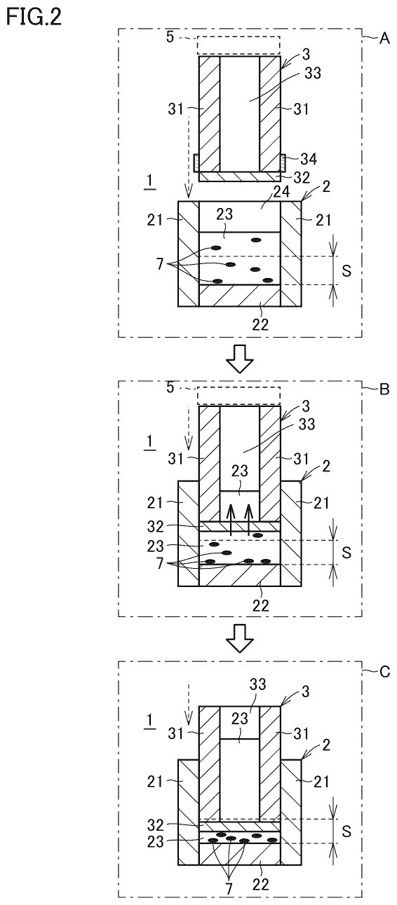

When the bacteria detection device 1 is used to perform the bacteria detection, how to specifically insert the storage member 3 into the reservoir 2 to detect the bacteria 7 will be described below with reference to FIG. 2.

The sensor 22 is constituted by an array sensor in which elements constituting a resonator are arranged adjacent to each other in a matrix. The array sensor may be constituted by CMOS, for example.

A detection signal of the sensor 22 is input to the analyzer 4. The analyzer 4 is constituted by a computer which includes a CPU (Central Processing Unit) 41, a memory 42 (any storage device such as a ROM (Read Only Memory), a RAM (Random Access Memory), a nonvolatile memory such as a flash memory, or the like), an input/output buffer (not shown) for inputting/outputting various signals, and the like.

The CPU 41 deploys a software program stored in a ROM such as a RAM, and executes the same. The ROM stores various programs describing a processing procedure related to analysis by the analyzer 4. The analyzer 4 operates in accordance with the program, and performs analysis related to the bacteria detection according to a detection signal input from the sensor 22. The analysis related to the bacteria detection is not limited to being processed by software, but may be processed by dedicated hardware (electronic circuit).

The analyzer 4 is connected to the input device 43 for inputting various kinds of data such as data used for various kinds of analysis related to the bacteria detection, and for inputting operation inputs required for executing various kinds of analysis related to the bacteria detection. The input device 43 includes various kinds of input devices such as a keyboard and a mouse.

The analyzer 4 is connected to the display 44 that displays various images such as analysis results of the bacteria detection. The display 44 includes various kinds of displays such as a liquid crystal display.

In response to the detection signal input from the sensor 22, the analyzer 4 analyzes the degree of growth of the bacteria 7 in the bacterial solution 23 according to the resonance frequency of each element in the array sensor constituting the sensor 22, and displays the analysis result on the display 44. Each element in the array sensor may be made to resonate at a frequency in a Gigahertz band, for example so as to detect the bacteria 7 in the bacterial solution 23. Each element in the array sensor may be made to resonate at a frequency in a Gigahertz band such as 30 GHz to 300 GHz. The preferable frequency may be, for example, around 60 GHz.

The analyzer 4 determines the growth of the bacteria 7 based on the fact that the dielectric constant of the water around the bacteria 7 in the bacterial solution 23 will change in response to the growth of the bacteria 7. For example, the analyzer 4 may acquire a change in the resonance frequency of each element of the array sensor constituting the sensor 22 in time series, and determine the degree of growth of the bacteria 7 in the bacterial solution 23 according to the change in the resonance frequency.

### [Operation States of Storage Member 3 During Bacteria Detection]

Next, when the bacteria detection device 1 is used to perform the bacteria detection, operation states of the storage member 3 will be described. FIG. 2 is a cross-sectional view illustrating operation states of the storage member 3 relative to the reservoir 2 during a bacteria detection by the bacteria detection device 1 according to the first embodiment.

The first state A illustrated in FIG. 2 is a state before the storage member 3 is inserted into the reservoir 2. The second state B illustrated in FIG. 2 is a state in which the storage member 3 is inserted into the reservoir 2 and is pushed downward by the weight of the pusher member 5, whereby the filter 32 is placed on the liquid surface of the bacterial solution 23 stored in the reservoir 2 to selectively permeate the liquid in the bacterial solution 23. The third state C illustrated in FIG. 2 is a state in which the storage member 3 is further inserted into the reservoir 2, whereby the filter 32 reaches the detection region S of the sensor 22.

With reference to FIG. 2, when the bacteria detection device 1 is used to perform the bacteria detection, firstly, in a state where the storage member 3 is not inserted into the reservoir 2 as illustrated in the first state A, the bacterial solution 23 containing the bacteria 7 is stored in the internal space 24 of the reservoir 2. In the first state A, the liquid level of the stored bacterial solution 23 is higher than the upper surface of the detection region S of the sensor 22. In this state, the bacteria 7 are also present in the bacterial solution 23 above the detection region S of the sensor 22.

Next, when the storage member 3 is inserted into the internal space 24 of the reservoir 2 and further pushed downward, the filter 32 is placed on the liquid surface of the bacterial solution 23 stored in the internal space 24 of the reservoir 2 as illustrated in the second state B. In the second state B, as indicated by arrows in the figure, the liquid in the bacterial solution 23 below the filter 32 selectively permeates through the filter 32, but the bacteria 7 below the filter 32 will not permeate through the filter 32. Thus, the filter 32 selectively permeates the liquid in the bacterial solution 23 through the openings, and the liquid in the bacterial solution 23 is selectively removed out of the detection region S of the sensor 22.

Then, when the storage member 3 is further pushed downward in the internal space 24 of the reservoir 2 as illustrated in the second state B, the filter 32 is also pushed downward, whereby the liquid in the bacterial solution 23 permeated through the filter 32 flows into the internal space 33 of the storage member 3 and is stored in the internal space 33.

Since the liquid in the bacterial solution 23 below the filter 32 is stored in the storage member 3, the amount of the bacterial solution 23 below the filter 32 decreases. As described above, as the filter 32 is pushed downward along with the storage member 3 in the internal space 24 of the reservoir 2, the storage amount of the bacterial solution 23 in the internal space 24 of the reservoir 2 decreases, and thereby the storage amount of the bacterial solution 23 in the internal space 33 of the storage member 3 increases.

Thereafter, when the filter 32 of the storage member 3 reaches the detection region S of the sensor 22 as illustrated in the third state C, the pusher member 5 in FIG. 1 is detached from the storage member 3, whereby the downward movement of the storage member 3 in the internal space 24 of the reservoir 2 is stopped. In the third state C, all of the bacteria 7 present in the bacterial solution 23 are condensed in the detection region S of the sensor 22. In this state, the sensor 22 is used to detect the bacteria 7 present in the bacterial solution 23.

As described above, when the storage member 3 is further pushed downward in the internal space 24 of the reservoir 2, the concentration of the bacteria 7 in the bacterial solution 23 is increased in the detection region S of the sensor 22. Since the sensor 22 is used to detect the bacteria 7 present in the bacterial solution 23 in a state where the concentration of the bacteria 7 in the bacterial solution 23 is increased in the detection region S of the sensor 22, the sensor can accurately detect the bacteria 7 in the bacterial solution 23.

The storage member 3 is not limited to being pushed downward by attaching the pusher member 5 to the storage member 3, the storage member 3 may be pushed downward at a constant speed by using a mechanical pushing device. The storage member 3 is not limited to being pushed downward by attaching the pusher member 5 to the storage member 3, the storage member 3 may be pushed downward at a constant speed by a human person. In addition, the storage member 3 may not be pushed downward at a constant speed, but may be pushed downward at a slower speed as the filter 32 approaches the detection region S of the sensor 22.

The arrival of the filter 32 of the storage member 3 to the detection region S may be detected by an optical sensor or by visual observation. In addition, the pusher member 5 may be detached by a human person or by using a mechanical device when it is detected that the filter 32 of the storage member 3 has reached the detection region S.

In addition, when the filter 32 reaches the detection region S of the sensor 22, the storage member 3 may be prevented from being pushed downward by providing a stopper member on the bottom of the internal space 24 of the reservoir 2 at a position to stop the filter 32 when the filter 32 reaches the detection region S.

### [Example Configuration of Bacteria Detection Device 1 Used to Perform Bacteria Detection on Plural Reservoirs 2]

Hereinafter, an example configuration in which the bacteria detection device 1 is used to perform the bacteria detection on a plurality of reservoirs 2 according to the first embodiment will be described.

When the bacteria detection device 1 is used to perform the bacteria detection on a plurality of reservoirs 2 such as a plurality of wells provided on a multi-well plate, the bacteria detection device 1 may be configured in such a manner that one filter 32 and one storage member 3 can be inserted into each reservoir 2.

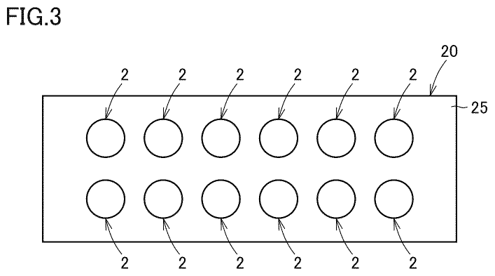

FIG. 3 is a plan view illustrating a multi-well plate 20 to be detected by the bacteria detection device 1 according to the first embodiment. In the multi-well plate 20, a plurality of reservoirs 2 are provided on a flat sheet member 25.

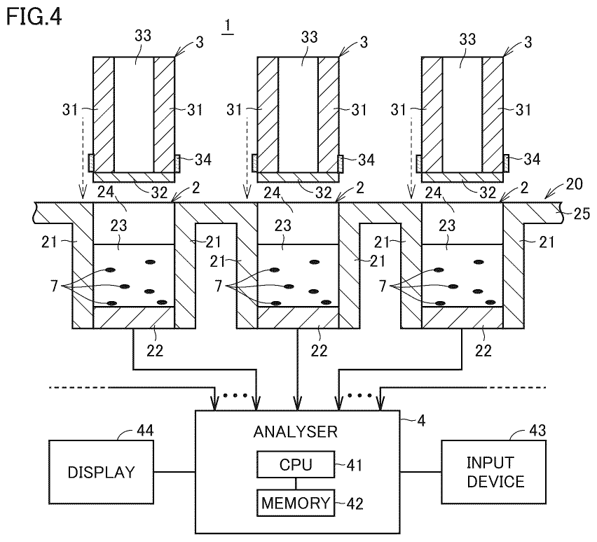

FIG. 4 is a diagram illustrating an example configuration in which the bacteria detection device 1 is used to perform the bacteria detection on a plurality of reservoirs 2 according to the first embodiment. FIG. 4 representatively illustrates some of the reservoirs 2 provided on the multi-well plate 20.

With reference to FIG. 4, when the bacteria detection device 1 is used to perform the bacteria detection on a plurality of reservoirs 2 according to the first embodiment, a plurality of storage members 3 are provided in such a manner that one storage member 3 can be inserted into each reservoir 2. Each reservoir 2 is provided with one sensor 22.

In the configuration of FIG. 4, when the bacteria detection is performed on a plurality of reservoirs 2, one storage member 3 is inserted into each reservoir 2 as illustrated in FIG. 2, and then the corresponding sensor 22 is used to detect the bacteria 7 in the bacterial solution 23. The insertion of the storage member 3 into each reservoir 2 and the detection of the bacteria 7 in each reservoir 2 by the sensor 22 may be performed on a plurality of reservoirs simultaneously or in a predetermined order.

The detection signals from a plurality of sensors 22 are input to the analyzer 4. In the analyzer 4, a program for analyzing bacteria in each reservoir 2 is stored in the ROM according to the detection signals input from the plurality of sensors 22, and by executing the program, the bacteria detection is performed on the bacterial solution 23 stored in each of the plurality of reservoirs 2.

According to the configuration illustrated in FIG. 4, the bacteria detection can be performed on a plurality of reservoirs 2.

### [Steps of Bacteria Detection Method by Bacteria Detection Device 1]

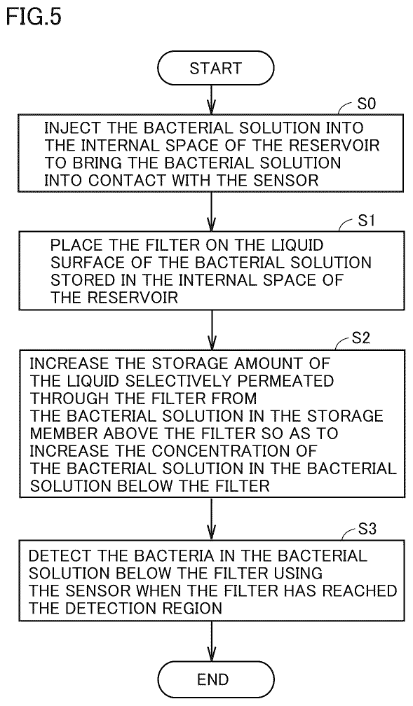

A bacteria detection method performed by the bacteria detection device 1 will be described with reference to FIG. 5. FIG. 5 is a flowchart illustrating steps of the bacteria detection method.

When the bacteria detection is performed by the bacteria detection device 1, firstly, in step S0, the bacterial solution 23 containing the bacteria 7 and the liquid is injected into the internal space 24 of the reservoir 2, whereby the bacterial solution 23 is brought into contact with the sensor 22. Thereafter, in step S1, the filter 32 is placed on the liquid surface of the bacterial solution 23 stored in the internal space 24 of the reservoir 2. Thus, the liquid in the bacterial solution 23 selectively permeates through the filter 32 and enters the internal space 33 of the storage member 3 on the filter 32, whereby the storage amount of the liquid from the bacterial solution 23 is increased in the storage member 3.

In step S2, by gradually pushing the storage member 3 and the filter 32 downward in the internal space 24 of the reservoir 2, the storage amount of the liquid selectively permeated through the filter 32 from the bacterial solution 23 is increased in the internal space 33 of the storage member 3 above the filter 32, whereby the concentration of the bacterial solution 7 in the bacterial solution 23 below the filter 32 is increased. As the liquid is selectively permeated through the openings of the filter 32, the liquid in the bacterial solution 23 below the filter 32 is selectively removed out of the detection region S of the sensor 22. Thus, as the liquid selectively permeated through the filter 32 from the bacterial solution 23 increases, the storage amount of the liquid from the bacterial solution 23 is increased in the internal space 33 of the storage member 3, while the storage amount of the bacterial solution 23 below the filter 32 is decreased.

In step S3, when the filter 32 reaches the detection region S of the sensor 22, the downward pushing of the storage member 3 and the filter 32 is stopped, and the sensor 22 is used to detect the bacteria 7 in the bacterial solution 23 below the filter 32.

By performing such a bacteria detection method, the bacteria 7 in the bacterial solution 23 is detected by the bacteria detection device 1 having a configuration as illustrated in FIGS. 1 and 4. According to such a bacteria detection method, since the bacteria 7 present in the bacterial solution 23 are detected by the sensor 22 after the concentration of the bacteria 7 in the bacterial solution 23 is increased in the detection region S of the sensor 22, the sensor can accurately detect the bacteria 7 in the bacterial solution 23. Further, since the concentration of the bacteria 7 in the bacterial solution 23 can be increased in the detection region S of the sensor 22, it is possible to detect the growth state of an overall small number of bacteria, which makes it possible to shorten the culture time.

### Second Embodiment

In the second embodiment, the description will be given to a bacteria detection device 1A in which the liquid selectively permeated through the filter 32 from the bacterial solution 23 is stored in a storage member 6 which is made of a liquid absorbing material and is provided above the filter 32, whereby the liquid selectively permeated through the filter 32 from the bacterial solution 23 is stored in the storage member 6.

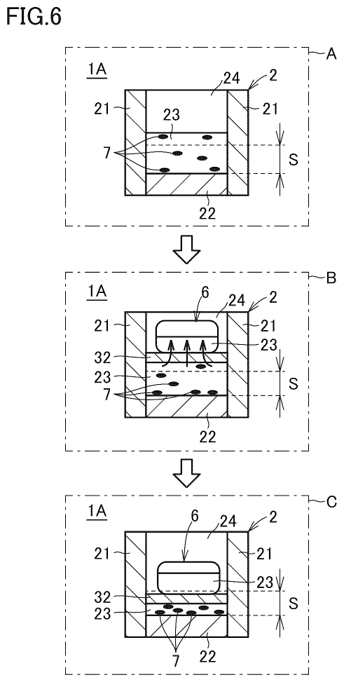

FIG. 6 is a cross-sectional view illustrating operation states of the storage member 6 relative to the reservoir 2 during a bacteria detection by the bacteria detection device 1A according to the second embodiment. The bacteria detection device 1A according to the second embodiment is different from the bacteria detection device 1 according to the first embodiment in that the storage member 6 is provided on an upper surface of the filter 32 to replace the storage member 3 provided on a lower bottom of the filter 32.

The storage member 6 is made of a liquid absorbing material, and is provided on the filter 32 in such a manner as to be placed on the filter 32. The filter 32 is attached to a frame that will not deform when the liquid in the bacterial solution 23 is permeated through the filter 32 and will not be deformed by the weight of the storage member 6 provided on the upper surface of the filter 32.

When the bacteria detection device 1A according to the second embodiment is used to perform the bacteria detection on a single reservoir 2, the sensor 22, the analyzer 4, the input device 43, and the display 44 are connected to each other as illustrated in FIG. 1. When the bacteria detection device 1A according to the second embodiment is used to perform the bacteria detection on a plurality of reservoirs 2, the sensor 22, the analyzer 4, the input device 43, and the display 44 are connected to each other as illustrated in FIG. 4.

The first state A illustrated in FIG. 6 is a state before the filter 32 and the storage member 6 are inserted into the reservoir 2. The second state B illustrated in FIG. 6 is a state in which the filter 32 and the storage member 6 are inserted into the internal space 24 of the reservoir 2, and the filter 32 is placed on the liquid surface of the bacterial solution 23 in the internal space 24 of the reservoir 2 to selectively permeate the liquid in the bacterial solution 23. The third state C illustrated in FIG. 6 is a state in which the filter 32 and the storage member 6 are further pushed downward in the internal space 24 of the reservoir 2, whereby the filter 32 reaches the detection region S of the sensor 22.

With reference to FIG. 6, when the bacteria detection device 1A is used to perform the bacteria detection, firstly, in the state where the filter 32 and the storage member 6 are not inserted into the internal space 24 of the reservoir 2 as illustrated in the first state A, the bacterial solution 23 containing the bacteria 7 and the liquid is stored in the internal space 24 of reservoir 2. In the first state A, the liquid level of the stored bacterial solution 23 is higher than the upper surface of the detection region S of the sensor 22. In this state, the bacteria 7 are present in the bacterial solution 23 above the detection region S of the sensor 22.

Next, the filter 32 and the storage member 6 are inserted into the internal space 24, whereby the filter 32 is placed on the liquid surface of the bacterial solution 23 stored in the reservoir 2 as illustrated in the second state B. In the second state B, as indicated by arrows in the figure, the liquid in the bacterial solution 23 selectively permeates through the filter 32, but the bacteria 7 will not permeate through the filter 32. Thus, when the filter 32 selectively permeates the liquid in the bacterial solution 23 through the openings, the liquid in the bacterial solution 23 is selectively removed out of the detection region S of the sensor 22.

Thus, the liquid selectively permeated through the filter 32 from the bacterial solution 23 is absorbed by the storage member 6, and is stored in the storage member 6. In FIG. 6, in order to clearly show the amount of the liquid absorbed by the storage member 6 from the bacterial solution 23, the region stored with the liquid from the bacterial solution 23 is denoted by a reference numeral 23.

The storage member 6 is configured to absorb the liquid from the bacterial solution 23 to a predetermined limit, and the weight of the storage member 6 increases as the amount of the liquid absorbed from the bacterial solution 23 increases. As the weight of the storage member 6 increases, the storage member 6 pushes the filter 32 downward by its own weight.

In the storage member 6, the liquid in the bacterial solution 23 below the filter 32 selectively permeates through the filter 32 and enters the storage member 6 until the absorption amount reaches the maximum absorption amount. The liquid in the bacterial solution 23 below the filter 32 is absorbed by the storage member 6, whereby the amount of the bacterial solution 23 stored below the filter 32 is decreased. Thereby, the storage member 6 pushes the filter 32 downward by its own weight in the internal space 24 of the reservoir 2.

Thereafter, when the filter 32 reaches the detection region S of the sensor 22 as illustrated in the third state C, the downward pushing of the filter 32 by the storage member 6 is stopped. The reason why the downward pushing of the filter 32 by the storage member 6 is stopped is that the absorption amount of the storage member 6 reaches the maximum absorption amount, and thereby the liquid in the bacterial solution 23 below the filter 32 cannot permeate through the filter 32 to enter the storage member 6.

Thus, the downward pushing of the filter 32 by the storage member 6 is stopped when the filter 32 reaches the detection region S of the sensor 22 may be realized by adjusting at least one of an initial storage amount of the bacterial solution 23 initially stored in the internal space 24 of the reservoir 2 and a maximum absorption amount of the storage member 6 so that the absorption amount of the storage member 6 reaches the maximum absorption amount when the filter 32 reaches the detection region S of the sensor 22 according to a relationship between the initial storage amount of the bacterial solution 23 and the maximum absorption amount of the storage member 6.

In the third state C, all of the bacteria 7 present in the bacterial solution 23 are condensed in the detection region S of the sensor 22. In this state, the sensor 22 is used to detect the bacteria 7 present in the bacterial solution 23.

As described above, when the filter 32 and the storage member 6 are pushed downward in the internal space 24 of the reservoir 2, the concentration of the bacteria 7 in the bacterial solution 23 is increased in the detection region S of the sensor 22. Since the sensor 22 is used to detect the bacteria 7 present in the bacterial solution 23 in a state where the concentration of the bacteria 7 in the bacterial solution 23 is increased in the detection region S of the sensor 22, the sensor 22 can accurately detect the bacteria 7 in the bacterial solution 23.

The arrival of the filter 32 of the storage member 6 to the detection region S may be detected by an optical sensor or by visual observation. In addition, when the filter 32 reaches the detection region S of the sensor 22, the storage member 6 may be prevented from being pushed downward by providing a stopper member on the bottom of the internal space 24 of the reservoir 2 at a position to stop the filter 32 when the filter 32 reaches the detection region S.

### [Steps of Bacteria Detection Method by Bacteria Detection Device 1A]

The steps of the bacteria detection method performed by the bacteria detection device 1A is the same as the steps illustrated in FIG. 5. The bacteria detection method by the bacteria detection device 1A will be described with reference to FIGS. 4 and 5.

When the bacteria detection is performed by the bacteria detection device 1A, firstly, in step S0, the bacterial solution 23 containing the bacteria 7 and the liquid is injected into the internal space 24 of the reservoir 2, whereby the bacterial solution 23 is brought into contact with the sensor 22. Thereafter, in step S1, the filter 32 and the storage member 6 are inserted in the internal space 24 of the reservoir 2, whereby the filter 32 is placed on the liquid surface of the bacterial solution 23. Thus, the liquid in the bacterial solution 23 selectively permeates through the filter 32 and is absorbed into the storage member 6 on the filter 32, whereby the storage amount of the liquid from the bacterial solution 23 is increased in the storage member 6.

In step S2, since the storage member 6 absorbs the liquid from the bacterial solution 23, which increases the weight of the storage member 6, the storage member 6 pushes the filter 32 downward by its own weight, whereby the storage amount of the liquid selectively permeated through the filter 32 from the bacterial solution 23 is increased in the storage member 6 on the filter 32, and thereby the concentration of the bacteria 7 in the bacterial solution 23 below the filter 32 is increased. As the liquid is selectively permeated through the openings of the filter 32, the liquid in the bacterial solution 23 below the filter 32 is selectively removed out of the detection region S of the sensor 22. Thus, as the liquid selectively permeated through the filter 32 from the bacterial solution 23 increases, the storage amount of the liquid absorbed by the storage member 6 from the bacterial solution 23 is increased, while the storage amount of the liquid in the bacterial solution 23 below the filter 32 is decreased.

In step S3, when the filter 32 reaches the detection region S of the sensor 22, the downward pushing of the filter 32 by the own weight of the storage member 6 is stopped, and the sensor 22 is used to detect the bacteria 7 in the bacterial solution 23 below the filter 32.

By performing such a bacteria detection method, the bacteria 7 in the bacterial solution 23 is detected by the bacteria detection device 1A having the configuration as illustrated in FIG. 6. According to such a bacteria detection method, since the bacteria 7 present in the bacterial solution 23 are detected by the sensor 22 after the concentration of the bacteria 7 in the bacterial solution 23 is increased in the detection region S of the sensor 22, the sensor can accurately detect the bacteria 7 in the bacterial solution 23. Further, since the concentration of the bacteria 7 in the bacterial solution 23 can be increased in the detection region S of the sensor 22, it is possible to detect the growth state of an overall small number of bacteria, which makes it possible to shorten the culture time.

The filter 32 may be placed on the liquid surface of the bacterial solution 23 in the internal space 24 of the reservoir 2 by a human person or by using a mechanical device. In addition, when the filter 32 reaches the detection region S of the sensor 22, the storage member 6 may be prevented from being pushed downward by its own weight by providing a stopper member on the bottom of the internal space 24 of the reservoir 2 at a position to stop the filter 32 when the filter 32 reaches the detection region S.

### <Drug Sensitivity Detection>

Hereinafter, an example drug sensitivity detection performed by the bacteria detection device 1 of the first embodiment or the bacteria detection device 1A of the second embodiment will be described.

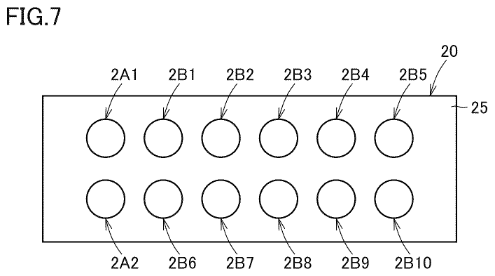

FIG. 7 is a plan view illustrating a multi-well plate 20 used in the drug sensitivity detection. The multi-well plate 20 illustrated in FIG. 7 has the same configuration as the multi-well plate 20 illustrated in FIG. 3.

In the case where a drug sensitivity detection is performed using the bacteria detection device 1 according to the first embodiment, a plurality of filters 32 and a plurality of storage members 3 are provided in such a manner that one filter 32 and one storage member 3 can be inserted into each reservoir 2. Each reservoir 2 is provided with one sensor 22 as described above. The detection signal of the sensor 22 is input to the analyzer 4 as illustrated in FIG. 4. The analyzer 4 performs an analysis process for the drug sensitivity detection according to the detection signal input from the sensor 22.

In the case where a drug sensitivity detection is performed using the bacteria detection device 1A according to the second embodiment, a plurality of filters 32 and a plurality of storage members 3 are provided in such a manner that one filter 32 and one storage member 6 can be inserted into each reservoir 2. Each reservoir 2 is provided with one sensor 22 as described above. The detection signal of the sensor 22 is input to the analyzer 4 as illustrated in FIG. 4. The analyzer 4 performs an analysis process for the drug sensitivity detection according to the detection signal input from the sensor 22.

With reference to FIG. 7, for example, in the case of performing the drug sensitivity detection, 2 wells, i.e., the reservoirs 2A1 and 2A2 are used as control wells, and 10 wells, i.e., the reservoirs 2B1 to 2B10 are used to culture bacteria in a bacterial solution containing an anti-bacteria agent.

In the reservoirs 2B1 to 2B10, bacteria are cultured in a bacterial solution preliminarily injected with different types of anti-bacteria agents or different concentrations of anti-bacteria agents in each reservoir. It should be noted that the anti-bacteria agent may be injected into each of the reservoirs 2B1 to 2B10 at any time while the bacteria are being cultured in the bacterial solution without the anti-bacteria agent.

When the drug sensitivity detection is performed in the configuration as illustrated in FIG. 7, the concentration of bacteria contained in the bacterial solution is increased in the detection region S of the sensor 22 as described above, whereby the sensor 22 can accurately detect the growth state of bacteria in the bacterial solution containing the anti-bacteria agent. Thus, in the configuration of this modification, the growth state of the bacteria in the bacterial solution containing the anti-bacteria agent can be accurately evaluated. Further, since the concentration of the bacteria 7 in the bacterial solution 23 can be increased in the detection region S of the sensor 22, it is possible to detect the growth state of an overall small number of bacteria, which makes it possible to shorten the time required for the drug sensitivity detection.

### <Example Detection Result>

Hereinafter, an example detection result of a bacteria detection performed by using the bacteria detection device 1 of the first embodiment or the bacteria detection device 1A of the second embodiment will be described. In the following, a detection result of a bacteria detection performed by using the bacteria detection device 1 of the first embodiment will be described as a representative example.

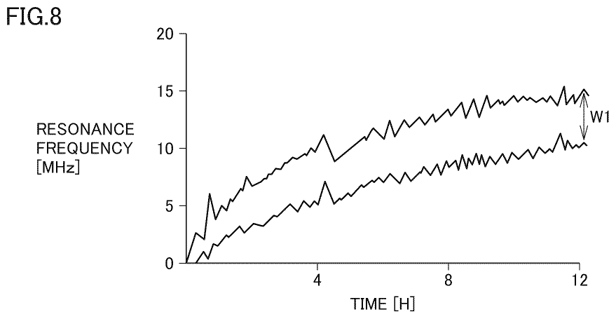

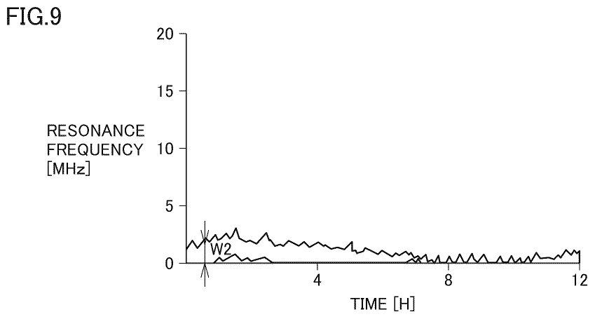

FIG. 8 is a graph illustrating a detection result when Mycobacterium tuberculosis is present in the bacterial solution. FIG. 9 is a graph illustrating a detection result when Mycobacterium tuberculosis is not present in the bacterial solution. In FIGS. 8 and 9, the vertical axis represents the resonance frequency (MHz), and the horizontal axis represents the elapsed time (H).

In order to clearly show changes in the resonance frequency over time of each element of the array sensor constituting the sensor 22, FIG. 8 and FIG. 9 each illustrate a representative example which shows changes in the resonance frequency over time of an element having the highest resonance frequency and changes in the resonance frequency over time of an element having the lowest resonance frequency. Therefore, in FIG. 8, the changes in the resonance frequency of the other elements are included in a range denoted by a width W1 in the figure. Similarly, in FIG. 9, the changes in the resonance frequency of the other elements are included in a range denoted by a width W2 in the figure. Thus, in FIG. 9, the changes in the resonance frequency of the other elements are included in a range denoted by a width W2 in the figure.

As illustrated in FIGS. 8 and 9, in the bacteria detection device 1, the sensor 22 can accurately detect the growth state of the bacteria 7 in the bacterial solution 23. Further, as illustrated in FIGS. 8 and 9, in the bacteria detection device 1, the sensor 22 can accurately detect the presence or absence of the bacteria 7 in the bacterial solution 23.

In the case where the bacteria detection is performed using the bacteria detection device 1A according to the second embodiment, the same detection results as the detection results illustrated in FIGS. 8 and 9 are obtained.

### [Modifications]

(1) In the first embodiment, it is described that the liquid selectively permeated through the filter 32 from the bacterial solution 23 is stored in the storage member 3 provided above the filter 32, and in the second embodiment, it is described that the liquid in the bacterial solution 23 selectively permeated the filter 32 is stored in the storage member 6 provided above the filter 32. However, the present invention is not limited thereto, and the liquid selectively permeated through the filter 32 from the bacterial solution 23 may be directly removed out of the reservoir 2 without being stored above the filter 32.

(2) In the first embodiment, it is described that the filter 32 is pushed by the storage member 3 in the reservoir 2 to reach the detection region S of the sensor 22, and in the second embodiment, it is described that the filter 32 is pushed by the storage member 6 in the reservoir 2 to reach the detection region S of the sensor 22. However, the present invention is not limited thereto, and the filter 32 may be fixed at a position, and the reservoir 2 may be moved upward to allow the filter 32 to reach the detection region S of the sensor 22.

(3) In the multi-well plate 20 illustrated in FIGS. 3 and 7, the sensor 22 is fixedly attached to the bottom of each of the plurality of reservoirs. However, the present invention is not limited thereto, and the sensor 22 may be detachably attached to the bottom of each of the plurality of reservoirs. Also, the sensor 22 of the single reservoir 2 illustrated in FIG. 1 may be detachably attached to the bottom of the reservoir.

(4) It is described that the filter 32 is a hydrophilic membrane filter. However, the present invention is not limited thereto, and any other type of filter may be used as long as the filter has a structure that allows the liquid in the bacterial solution 23 to permeate therethrough but does not allow the bacteria 7 to permeate therethrough.

(5) It is described that the bacteria detection device 1 or 1A is used to detect the bacteria 7 with the filter 32 reaching the detection region S of the sensor 22. However, the present invention is not limited thereto, and if the concentration of the bacteria 7 in the detection region S of the sensor 22 may be improved by pushing the filter 32 downward, the bacteria detection device 1 or 1A may be used to detect the bacteria 7 by stopping the pushing of the filter 32 before the filter 32 reaches the detection region S of the sensor 22. Further, the bacteria detection device 1 or 1A may be used to detect the bacteria 7 by stopping the pushing of the filter 32 after the filter 32 enters the detection region S of the sensor 22.

(6) It is described that the reservoir 2 has a cylindrical shape. However, the reservoir 2 may be cylindrical, triangular in cross section, or square in cross section. In this case, the shape of the filter 32 and the shape of the storage member 3 in the first embodiment and the shape of the filter 32 in the second embodiment may be modified in response to the inner shape of the reservoir 2.

### [Aspect]

As described above, the present embodiment includes the following aspects.

### [Aspect 1]

A bacteria detection device (bacteria detection device 1, bacteria detection device 1A) includes:
a reservoir (reservoir 2) which stores a bacterial solution (bacterial solution 23) containing bacteria (bacteria 7) and liquid;
a sensor (sensor 22) which is provided on a bottom of the reservoir (reservoir 2) and configured to detect the bacteria (bacteria 7) contained in the bacterial solution (bacterial solution 23) stored in the reservoir (reservoir 2); and
a filter (filter 32) which is placed on a liquid surface of the bacterial solution (bacterial solution 23) stored in the reservoir (reservoir 2) and has an opening for selectively permeating the liquid in the bacterial solution (bacterial solution 23),
the filter (filter 32) is configured to selectively permeate the liquid in the bacterial solution (bacterial solution 23), thereby increasing the concentration of the bacteria (bacteria 7) contained in the bacterial solution (bacterial solution 23) in a detection region (detection region S) of the sensor (sensor 22).

According to this aspect, since the liquid in the bacterial solution (bacterial solution 23) is selectively removed out of the detection region (detection region S) of the sensor (sensor 22) by selectively permeating the liquid in the bacterial solution (bacterial solution 23) through the filter (filter 32), whereby the concentration of the bacteria (bacteria 7) contained in the bacterial solution (bacterial solution 23) is increased in the detection region (detection region S) of the sensor (sensor 22), the bacteria (bacteria 7) in the bacterial solution (bacterial solution 23) can be accurately detected.

### [Aspect 2]

The bacteria detection device (bacteria detection device 1, bacteria detection device 1A) according to aspect 1 further includes a storage member (storage members 3, 6) which is provided above the filter (filter 32) and configured to store the liquid selectively permeated through the filter (filter 32) from the bacterial solution (bacterial solution 23).

According to this aspect, since the liquid selectively permeated through the filter (filter 32) from the bacterial solution (bacterial solution 23) is store in the storage member (storage member 3 or 6) provided above the filter (filter 32), the liquid selectively permeated through the filter (filter 32) from the bacterial solution (bacterial solution 23) can be retained above the filter (filter 32).

### [Aspect 3]

In the bacteria detection device (bacteria detection device 1) according to aspect 2, the storage member (storage member 3) is a cylindrical body, the filter (filter 32) is provided on a bottom of the storage member (storage member 3), and the storage member (storage member 3) stores the liquid selectively permeated through the filter (filter 32) from the bacterial solution (bacterial solution 23) inside the cylindrical body.

According to this aspect, since the liquid selectively permeated through the filter (filter 32) from the bacterial solution (bacterial solution 23) is stored inside the cylindrical body, the liquid selectively permeated through the filter (filter 32) from the bacterial solution (bacterial solution 23) can be stored above the filter (filter 32).

### [Aspect 4]

In the bacteria detection device (bacteria detection device 1A) according to aspect 2, the storage member (storage member 6) is a liquid absorbing material, the storage member is placed on the filter (filter 32), and is configured to store the liquid selectively permeated through the filter (filter 32) from the bacterial solution (bacterial solution 23) by absorbing the liquid into the liquid absorbing material.

According to this aspect, since the liquid selectively permeated through the filter (filter 32) from the bacterial solution (bacterial solution 23) is absorbed by the liquid absorbing material placed on the filter (filter 32), the liquid selectively permeated through the filter (filter 32) from the bacterial solution (bacterial solution 23) can be retained in the liquid absorbing material placed on the filter (filter 32).

### [Aspect 5]

In the bacteria detection device (bacteria detection device 1, bacteria detection device 1A) according to any one of aspects 1 to 4, the filter (filter 32) is a hydrophilic membrane filter.

According to this aspect, it is possible for the bacterial solution (bacterial solution 23) to easily permeate through the filter (filter 32).

### [Aspect 6]

In the bacteria detection device (bacteria detection device 1, bacteria detection device 1A) according to any one of aspects 1 to 5, the bacterial solution (bacterial solution 23) stored in the reservoir (reservoirs 2B1 to 2B10) further contains an anti-bacteria agent.

According to this aspect, the bacterial solution (bacterial solution 23) stored in the reservoirs (reservoirs 2B1 to 2B10) can be subjected to drug sensitivity detection.

### [Aspect 7]

A bacteria detection method includes:
a step (step S0) of bringing a bacterial solution (bacterial solution 23) containing bacteria (bacteria 7) and liquid into contact with a sensor configured to detect the bacteria; and
a step (step S2) of selectively removing the liquid in the bacterial solution (bacterial solution 23) out of a detection region (detection region S) of the sensor (sensor 22) by using a filter (filter 32) having an opening to selectively permeate the liquid in the bacterial solution (bacterial solution 23) so as to increase the concentration of the bacteria (bacterial 7) contained in the bacterial solution (bacterial solution 23) in the detection region (detection region S) of the sensor (sensor 22).

According to this aspect, since the liquid in the bacterial solution (bacterial solution 23) is selectively removed out of the detection region (detection region S) of the sensor (sensor 22) by selectively permeating the liquid in the bacterial solution (bacterial solution 23) through the filter (filter 32), the concentration of the bacteria (bacteria 7) contained in the bacterial solution (bacterial solution 23) is increased in the detection region (detection region S) of the sensor (sensor 22), the bacteria (bacteria 7) in the bacterial solution (bacterial solution 23) can be accurately detected.

### [Aspect 8]

The bacteria detection method according to aspect 7 further includes a step (step S3) of using the sensor (sensor 22) to detect the bacteria (bacteria 7) contained in the bacterial solution (bacterial solution 23) with the filter (filter 32) reaching the detection region (detection region S) of the sensor (sensor 22).

According to this aspect, since the sensor (sensor 22) detects the bacteria (bacteria 7) contained in the bacterial solution (bacterial solution 23) with the filter (filter 32) reaching the detection region (detection region S) of the sensor (sensor 22), the bacteria (bacteria 7) in the bacterial solution (bacterial solution 23) can be more accurately detected.

It should be understood that the embodiments and the examples disclosed herein have been presented for the purpose of illustration and description but not limited in all aspects. It is intended that the scope of the present invention is not limited to the description above but defined by the scope of the claims and encompasses all modifications equivalent in meaning and scope to the claims.

What is claimed is:

1. A bacteria detection device comprising:
a reservoir which stores a bacterial solution containing bacteria and liquid;
a sensor which is provided on a bottom of the reservoir and configured to detect the bacteria contained in the bacterial solution stored in the reservoir; and
a filter which is placed on a liquid surface of the bacterial solution stored in the reservoir and has an opening for selectively permeating the liquid in the bacterial solution,
the filter being configured to selectively permeate the liquid in the bacterial solution, thereby increasing the concentration of the bacteria contained in the bacterial solution in a detection region of the sensor.

2. The bacteria detection device according to claim 1, further comprising:
a storage member which is provided above the filter and configured to store the liquid selectively permeated through the filter from the bacterial solution.

3. The bacteria detection device according to claim 2, wherein
the storage member is a cylindrical body,
the filter is provided on a bottom of the storage member, and
the storage member stores the liquid selectively permeated through the filter from the bacterial solution inside the cylindrical body.

4. The bacteria detection device according to claim 2, wherein
the storage member is made of a liquid absorbing material,
the storage member is placed on the filter, and is configured to store the liquid selectively permeated through the filter from the bacterial solution by absorbing the liquid into the liquid absorbing material.

5. The bacteria detection device according to any one of claims 1 to 4, wherein
the filter is a hydrophilic membrane filter.

6. The bacteria detection device according to any one of claims 1 to 4, wherein
the bacterial solution stored in the reservoir further contains an anti-bacteria agent.

7. A bacteria detection method comprising:
bringing a bacterial solution containing bacteria and liquid into contact with a sensor configured to detect the bacteria; and
selectively removing the liquid in the bacterial solution out of a detection region of the sensor by using a filter having an opening to selectively permeate the liquid in the bacterial solution so as to increase the concentration of the bacteria contained in the bacterial solution in the detection region of the sensor.

8. The bacteria detection method according to claim 7, further comprising:
using the sensor to detect the bacteria contained in the bacterial solution with the filter reaching the detection region of the sensor.
